(19) United States
(12) Patent Application Publication
(10) Pub. No.: US 2025/0057652 A1
(43) Pub. Date: Feb. 20, 2025

(54) PROSTHETIC MITRAL VALVE WITH IMPROVED ATRIAL AND/OR ANNULAR APPOSITION AND PARAVALVULAR LEAKAGE MITIGATION

(71) Applicant: 4C Medical Technologies, Inc., Maple Grove, MN (US)

(72) Inventors: Saravana B. Kumar, Minnetonka, MN (US); Steven D. Kruse, Maple Grove, MN (US); Jeffrey R. Stone, Minnetonka, MN (US)

(21) Appl. No.: 18/748,331

(22) Filed: Jun. 20, 2024

Related U.S. Application Data
(63) Continuation of application No. 17/163,910, filed on Feb. 1, 2021, now Pat. No. 12,053,375.
(60) Provisional application No. 62/985,411, filed on Mar. 5, 2020.

(51) Int. Cl. A61F 2/24

(52) U.S. Cl. CPC A61F 2/2427; A61F 2/2418; A61F 2250/0048; A61F 2250/0056

(57) ABSTRACT

The present invention provides a prosthetic heart valve device with improved fit and/or apposition between the device frame and left atrial tissue and/or the device base and the annular tissue of the left atrium to improve shifting of the implanted device and/or mitigate paravalvular leakage. The improved fit and/or apposition arises in various embodiments by providing or allowing an asymmetrical frame and/or frame base and/or providing a lower lip to aid in conforming to the asymmetrical shape of the atrium and/or ensure firm positioning therein. An additional benefit of these arrangement(s) is mitigation of paravalvular leakage as a result of improved fit and seal. In certain embodiments, the asymmetry of the frame assists with delivery of the device into the atrium.

### CROSS-REFERENCE TO RELATED APPLICATIONS

This application claims priority to provisional application No. 62/985,411, filed Mar. 5, 2020 and titled PROSTHETIC MITRAL VALVE WITH IMPROVED ATRIAL AND/OR ANNULAR APPOSITION AND PARAVALVULAR LEAKAGE MITIGATION, the contents of which are incorporated hereto in its entirety.

### STATEMENT REGARDING FEDERALLY SPONSORED RESEARCH OR DEVELOPMENT

Not Applicable

### BACKGROUND OF THE INVENTION

### Field of the Invention

The invention relates to devices and methods for implanting devices within a heart chamber. More specifically, the invention relates to improved apposition of the implanted prosthetic heart valve with the left atrium and/or the annulus as well as improving paravalvular leakage and improved delivery and/or recapture.

### Description of the Related Art

The human heart comprises four chambers and four heart valves that assist in the forward (antegrade) flow of blood through the heart. The chambers include the left atrium, left ventricle, right atrium and right ventricle. The four heart valves include the mitral valve, the tricuspid valve, the aortic valve and the pulmonary valve. See generally FIG. 1.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood into the chamber it just flowed out of. For example, the mitral valve when working properly provides a one-way valving between the left atrium and the left ventricle, opening to allow antegrade flow from the left atrium to the left ventricle and closing to prevent retrograde flow from the left ventricle into the left atrium. This retrograde flow, when present, is known as mitral regurgitation or mitral valve regurgitation.

FIG. 2 illustrates the relationship between the left atrium, annulus, chordae tendineae and the left ventricle relative to the mitral valve leaflets. As is shown, the upper surface of the annulus forms at least a portion of the floor or lower surface of the left atrial chamber, so that for purposes of description herein, the upper surface of the annulus is defined as marking the lower boundary of the left atrial chamber.

Native heart valves may be, or become, dysfunctional for a variety of reasons and/or conditions including but not limited to disease, trauma, congenital malformations, and aging. These types of conditions may cause the valve structure to fail to close properly resulting in regurgitant retrograde flow of blood from the left ventricle to the left atrium in the case of a mitral valve failure. FIG. 3 illustrates regurgitant blood flow with an exemplary dysfunctional mitral valve.

Mitral valve regurgitation is a specific problem resulting from a dysfunctional mitral valve that allows at least some retrograde blood flow back into the left atrium from the right atrium. In some cases, the dysfunction results from mitral valve leaflet(s) that prolapse up into the left atrial chamber, i.e., above the upper surface of the annulus as designated by line or plane A, instead of connecting or coapting to block retrograde flow. This backflow of blood places a burden on the left ventricle with a volume load that may lead to a series of left ventricular compensatory adaptations and adjustments, including remodeling of the ventricular chamber size and shape, that vary considerably during the prolonged clinical course of mitral regurgitation.

Regurgitation can be a problem with native heart valves generally, including tricuspid. aortic and pulmonary valves as well as mitral valves.

Native heart valves generally, e.g., mitral valves, therefore, may require functional repair and/or assistance, including a partial or complete replacement. Such intervention may take several forms including open heart surgery and open heart implantation of a replacement heart valve. See e.g., U.S. Pat. No. 4,106,129 (Carpentier), for a procedure that is highly invasive, fraught with patient risks, and requiring not only an extended hospitalization but also a highly painful recovery period.

Less invasive methods and devices for replacing a dysfunctional heart valve are also known and involve percutaneous access and catheter-facilitated delivery of the replacement valve. Most of these solutions involve a replacement heart valve attached to a structural support such as a stent, commonly known in the art, or other form of wire network designed to expand upon release from a delivery catheter. Sec, e.g., U.S. Pat. No. 3,657,744 (Ersck); U.S. Pat. No. 5,411,552 (Andersen). The self-expansion variants of the supporting stent assist in positioning the valve, and holding the expanded device in position, within the subject heart chamber or vessel. This self-expanded form also presents problems when, as is often the case, the device is not properly positioned in the first positioning attempt and, therefore, must be recaptured and positionally adjusted. This recapturing process in the case of a fully, or even partially, expanded device requires re-collapsing the device to a point that allows the operator to retract the collapsed device back into a delivery sheath or catheter, adjust the inbound position for the device and then re-expand to the proper position by redeploying the positionally-adjusted device distally out of the delivery sheath or catheter. Collapsing the already expanded device is difficult because the expanded stent or wire network is generally designed to achieve the expanded state which also resists contractive or collapsing forces.

Besides the open heart surgical approach discussed above, gaining access to the valve of interest is achieved percutaneously via one of at least the following known access routes: transapical; transfemoral; transatrial; and transseptal delivery techniques.

Transseptal delivery involves creating an access hole in the septum between the right and left atria. Once the delivery and implantation of the prosthetic heart valve device is achieved, the septal hole either remains open to heal on its own, or is sealed at least partially. Transseptal delivery, as well as the other delivery techniques, may require recapture of an at least partially deployed or expanded device within the distal lumen of the delivery catheter in order to reposition and/or reorient the device before delivering, expanding, positioning and implanting the device.

Generally, the art is focused on systems and methods that, using one of the above-described known access routes, allow a partial delivery of the collapsed valve device, wherein one end of the device is released from a delivery sheath or catheter and expanded for an initial positioning followed by full release and expansion when proper positioning is achieved. See, e.g., U.S. Pat. Nos. 8,852,271 (Murray, III); U.S. Pat. No. 8,747,459 (Nguyen); U.S. Pat. No. 8,814,931 (Wang); U.S. Pat. No. 9,402,720 (Richter); 8,986,372 (Murray, III); and 9,277,991 (Salahich); and U.S. Pat. Pub. Nos. 2015/0272731 (Racchini); and 2016/0235531 (Ciobanu).

In addition, all known prosthetic heart valves are intended for full replacement of the native heart valve. Therefore, these replacement heart valves, and/or anchoring or tethering structures, physically extend out of the left atrial chamber, in the case of mitral valves, and engage the inner annulus and/or valve leaflets, in many cases pinning the native leaflets against the walls of the inner annulus, thereby permanently eliminating all remaining functionality of the native valve and making the patient completely reliant on the replacement valve. In other cases, the anchoring structures extend into the left ventricle and may anchor into the left ventricle wall tissue and/or the sub-annular surface at the top of the left ventricle. Others may comprise a presence in, or engagement with, a pulmonary artery.

Obviously, there will be cases when native valve has lost virtually complete functionality before the interventional implantation procedure. In this case the preferred solution will comprise an implant that does not extent outside of, e.g., the left atrium, and that functions to completely replace the native valve function. However, in many other cases, the native valve remains functional to an extent and may, or may not, continue to lose functionality after the implantation procedure. A preferred solution in this case comprises delivery and implantation of a valve device that will function both as a supplemental or augmentation valve without damaging the native leaflets in order to retain native valve leaflet functionality as long as present, while also being fully capable of replacing the native function of a valve that slowly loses most or all of its functionality post-implantation of the prosthetic valve.

In all cases, including two-chamber solutions, paravalvular leakage (PVL) may develop as a result of insufficient sealing or apposition of the prosthetic valve device and the native chamber tissue, including but not limited to annular sealing which, in some cases, may result from movement of the implanted device within the heart chamber. In the case of the exemplary mitral valve, PVL results in a retrograde leak of blood from the left ventricle to the left atrium, reducing the efficiency of the heart. Lack of sealing apposition may occur for several reasons.

For example, patients may have at least some calcification in the heart chamber, particularly in the annular surface which works to reduce compliance of that calcified tissue. This reduced compliance reduces the ability of the tissue and the prosthetic heart valve device to seal together on implantation, leaving gaps between tissue and device. The mitral valve annulus and the tricuspid valve annulus may be affected by calcification, leading to poor sealing apposition with the implanted prosthetic heart valve device and PVL.

As shown in the Figures, the left atrium further comprises an asymmetric shape which can result in suboptimal apposition by the device's frame against the atrial walls and/or tissue and/or the device's base against the annulus.

Certain inventive embodiments described herein are readily applicable to single or two chamber solutions, unless otherwise indicated. Moreover, certain embodiments discussed herein may be applied to preservation and/or replacement of native valve functionality generally, with improved apposition and/or PVL mitigation and/or delivery/recapture, and are not, therefore, limited to the mitral valve and may be extended to include devices and methods for treating the tricuspid valve, the aortic valve and/or pulmonary valves.

Various embodiments of the several inventions disclosed herein address these, inter alia, issues.

### BRIEF SUMMARY OF THE INVENTION

The present invention provides a prosthetic heart valve device with improved fit and/or apposition between the device frame and left atrial tissue and/or the device base and the annular tissue of the left atrium to improve shifting of the implanted device and/or mitigate paravalvular leakage. The improved fit and/or apposition arises in various embodiments by providing or allowing an asymmetrical frame and/or frame base and/or providing a lower lip to aid in conforming to the asymmetrical shape of the atrium and/or ensure firm positioning therein. An additional benefit of these arrangement(s) is mitigation of paravalvular leakage as a result of improved fit and seal. In certain embodiments, the asymmetry of the frame assists with delivery of the device into the atrium.

### BRIEF DESCRIPTION OF THE SEVERAL VIEWS OF THE DRAWINGS

FIG. 1 illustrates certain features of the heart in cross-section.

FIG. 2 illustrates a cross-sectional perspective view of the left side of the heart.

FIG. 3 illustrates a cross-sectional view of the heart showing retrograde blood flow resulting from mitral valve regurgitation compared with normal blood flow.

FIG. 4A illustrates a partial cutaway side view of one embodiment of the present invention.

FIG. 4B illustrates a side cutaway view of one embodiment of a valve support.

FIG. 5 illustrates a partial cutaway side view of one embodiment of the present invention.

FIG. 6 illustrates a partial cutaway side view of one embodiment of the present invention.

FIG. 7 illustrates a partial cutaway side view of one embodiment of the present invention.

FIG. 8 illustrates a partial cutaway side view of one embodiment of the present invention.

FIG. 9 illustrates a partial cutaway side view of one embodiment of the present invention.

FIG. 10 illustrates a partial cutaway side view of one embodiment of the present invention.

FIG. 11A illustrates a partial cutaway side view of one embodiment of the present invention.

FIG. 11B illustrates a top cutaway view of one embodiment of the present invention.

FIG. 12 illustrates a side cutaway view of one embodiment of the present invention.

FIG. 13 illustrates a side cutaway view of one embodiment of the present invention.

FIG. 14 illustrates a side cutaway view of one embodiment of the present invention.

FIG. 15 illustrates a side cutaway view of one embodiment of the present invention.

FIG. 16 illustrates a side view of one embodiment of the present invention.

FIG. 17 illustrates a side view of one embodiment of the present invention.

FIG. 18A illustrates a cutaway view of one embodiment of a mandrel of the present invention.

FIG. 18B illustrates a side view of one embodiment of the present invention.

FIG. 19A illustrates a cutaway view of one embodiment of a mandrel of the present invention.

FIG. 19B illustrates a side view of one embodiment of the present invention.

FIG. 20A illustrates a cutaway view of one embodiment of a mandrel of the present invention.

FIG. 20B illustrates a side view of one embodiment of the present invention.

FIG. 21 illustrates a side view of one embodiment of the present invention.

### DETAILED DESCRIPTION OF THE INVENTION

Various embodiments of the present invention comprise a prosthetic heart valve anchoring solution that combines improved PVL mitigation through improved sealing and/or apposition between the implanted device and the heart chamber tissue.

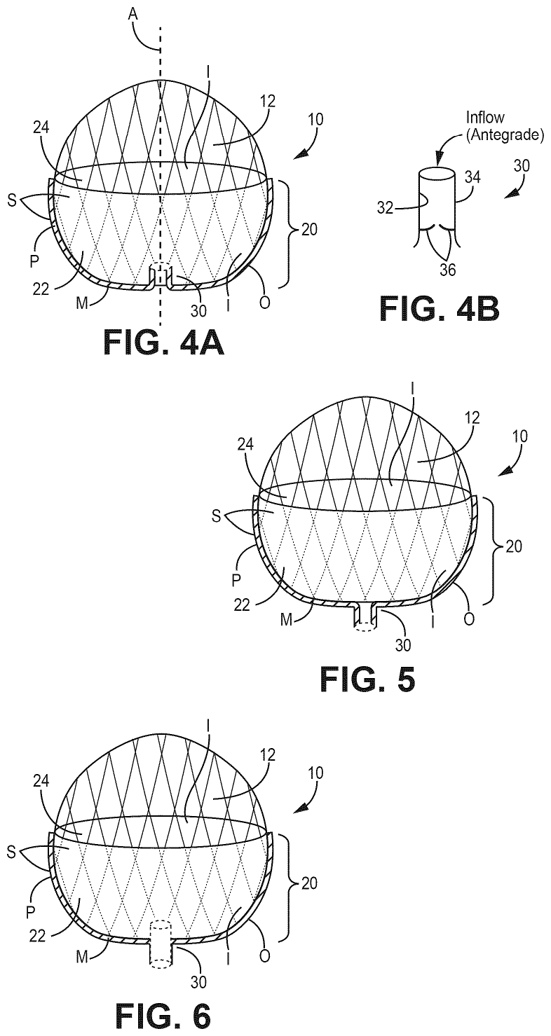

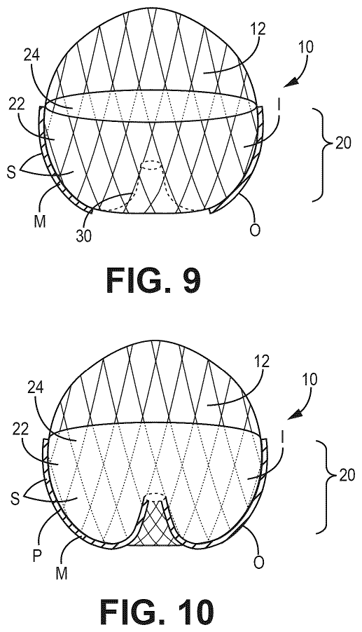

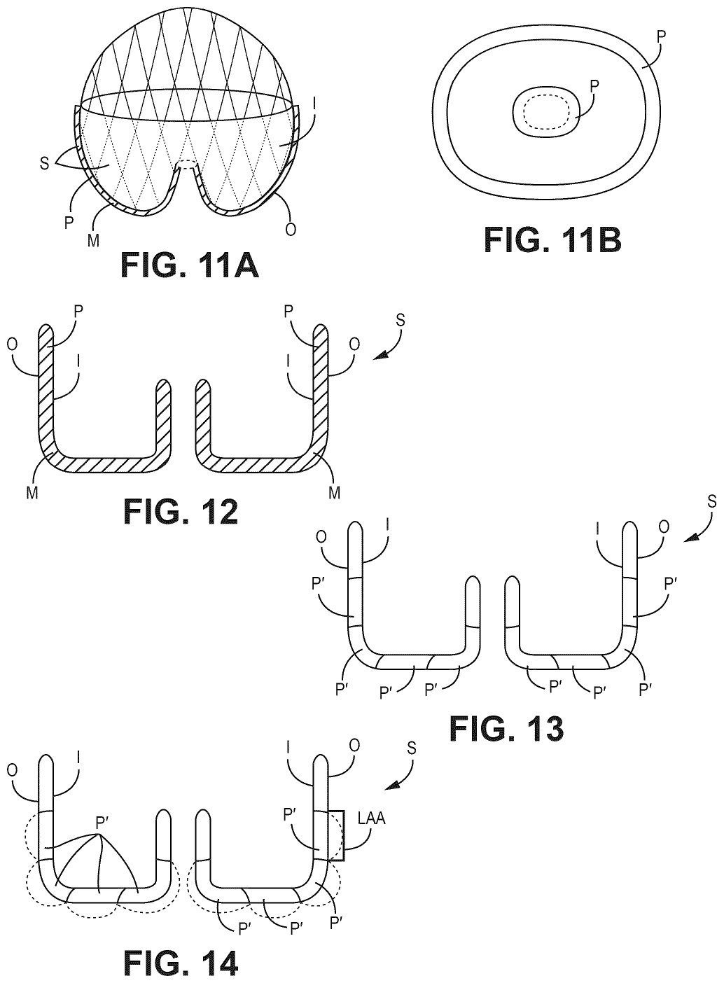

The invention will be described in the context of an exemplary single-chamber expanded and implanted device structure comprises certain exemplary embodiments as shown in FIGS. 4A-11B and 16-17. As stated above, however, the various embodiments of the invention extend to implanted prosthetic heart valve devices generally including but not limited to 1 and/or 2-chamber solutions. Moreover, various embodiments of the invention are described as relating to prosthetic mitral valve devices. However, it will be readily appreciated by the skilled artisan that these embodiments may be applied to supplement and/or replace the functionality of heart valves generally.

With specific reference to FIGS. 4A-6, exemplary embodiments of a collapsible, and expandable, anchoring structure 10 comprising an expandable stent frame 12, or other expandable material such as a wire mesh and/or a shape memory metal or polymer or the equivalent comprising an expandable and collapsible web or interconnected cells as is known in the art. Anchoring structure 10 preferably may be biased to expand to achieve the expanded state from a collapsed state, though other collapsed-to-expanded mechanisms may also be employed. Further, anchoring structure 10 may comprise a base section 20 that may be formed of the expandable stent frame 12 or equivalent and, therefore may achieve a plurality of expanded states in order to expand and contract with the natural movements of the heart chamber walls and the annulus, including the upper annular surface and/or portions of the inner throat of the annulus located between the upper annular surface or floor of the exemplary left atrium and the left ventricle.

Base section 20 comprises an outer surface 22 and an inner surface 24 and comprises a valve support 30 either integrally formed from the base section or operatively engaged or otherwise attached to base section. Valve support 30 comprises an inner surface 32 and an outer surface 34 wherein valve support 30 is adapted to substantially align with the subject annulus and allow one-way, antegrade blood flow therethrough while preventing retrograde blood flow as a result of prosthetic leaflet(s) 36 disposed on the inner surface 32 of valve support 30.

Valve support 30 may be disposed wholly or at least partially within base section 20 or may, in alternate embodiments, completely extend away from base section 20 with no part of valve support 30 within the base section 20. Thus, as shown in FIG. 4A, valve support 30 is disposed entirely within base section 20. FIG. 5 illustrates the valve support 30 extending generally entirely outside base section 20. FIG. 6 illustrates the valve support 30 partially within base section 20 and also extending away from, and outside of, base section 20. FIG. 8 illustrates a variation of the valve support 30 of FIG. 4A in that the valve support 30 is formed integrally with base section 20.

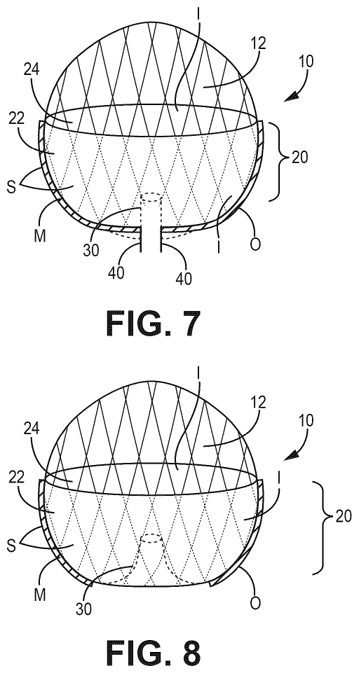

Reference is now made to exemplary boss structure 40 shown in FIG. 7, which when present may be used to align the anchoring structure 10 in the annulus and, in combination with the base section 20, assist with sealing apposition against portions of the annulus including the annular surface and inner throat of the annulus within which boss 40 extends. Boss 40 may be used in combination with any of the structures described herein including, as shown attached to or integrally formed from base section 20 and aligned with, and/or effectively extending, the flow channel defined within valve support 30. In this sense, boss 40 is similar to the extended valve support 30 of FIG. 6, but the boss 40 does not support prosthetic leaflets therein. In other embodiments, portions of the anchoring structure, including the valve support 30 and/or boss 40, may extend downstream in the antegrade direction away from the upper surface of the annulus into the inner throat of the annulus. In some cases, the structure may extend downward to pin the native leaflets. In other cases, the structure may extend downward into the annulus but stopping short of pinning the native leaflets.

Other variations of prosthetic valve devices are known in the art and will also benefit from variations of the present invention.

It is known to cover at least a portion, typically the lower outer portion, of an anchoring frame for a prosthetic heart valve with some fabric or tissue to help prevent PVL. Known embodiments create bunching and the like of the covering material to form a seal against PVL. These solutions however do not properly solve the fit and/or apposition problems arising from annular calcification and/or the varying and variety of the annular landscape.

Thus, with reference to the Figures, base section's outer surface 22 may be at least partially covered with a skirt S formed from, or comprising, a material M that conforms and seals with portions of the atrial wall and/or the upper annular surface. In some embodiments as illustrated, portions of the anchoring structure 10 and/or the valve support 30 may extend a distance into the annular throat, i.e., below the annular surface toward the native leaflets, wherein at least some of the anchoring structure 10 and/or valve support 30 may be covered with material M.

In some embodiment, the material M may seal with at least part of the circumferential region of the wall that encompasses the left atrial appendage (LAA) within the exemplary left atrium in order to seal the LAA.

Material M may comprise a substance or compound that is hydrophilic, wherein a skirt for base section 20 may be formed, in whole or in part, from at least material M and also be hydrophilic. In this case, the hydrophilic skirt may absorb water from the patient's blood and expand or swell to provide a tightened seal and/or apposition between the base section 20 and relevant regions of the heart chamber, thereby serving as a barrier to retrograde blood flow upon implantation, mitigating and/or preventing PVL.

The hydrophilic material M may comprise a hydrophilic gel and/or hydrophilic polymer, for example that can be selected with a swelling modulus, or more than one swelling modulus, to help ensure that the swollen material M and/or hydrophilic skirt comprising material M swells to the “right” size and further ensure that the swelling process occurs slowly and gently to allow for the device to be properly positioned in the heart chamber before substantial swelling occurs. An exemplary hydrophilic hydrogel may comprise poly (vinyl alcohol) (PVA).

The hydrogel embodiment of material M may comprise hydrophilic polymer(s) that have been chemically, physically and/or ionically crosslinked to form a matrix that swells in water. The degree of swelling of hydrogels in water is determined by a balance between the free energy of polymer/solvent mixing, ionic interactions and elastic forces and is influenced by the extent of crosslinking and the chemical nature of the polymer. The degree of swelling, in turn, determines the mesh size of the hydrogel. Hydrophilic hydrogels and/or polymers may be temperature responsive and/or pH-responsive. Some, such as chitosan and alginate are naturally occurring and offer both natural hydrophilicity and biocompatibility. Still further, swelling may be initiated by mechanical means such as agitation.

Other hydrophilic materials such as hydrophilic metals may comprise portions of the anchoring structure 10.

The hydrophilic material M may be encapsulated within easily breakable, or dissolvable or biodegradable or bioerodable nanoparticles, wherein when the nanoparticles are broken, the hydrophilic material M is exposed to water and begin the swelling process. In this case, the prosthetic heart valve device will be positioned and implanted before any substantial swelling can occur.

Skirt S may comprise two layers of material, an inner layer I attached to the outer surface 22 of base section 20, and an outer layer O wherein the inner and outer layer form a pocket or a series of pockets P. The hydrophilic material M may be disposed or attached or incorporated at designed areas within the pocket or series of pockets to facilitate swelling at the interfacing regions between the expanded device and the patient's anatomy that are most vulnerable to PVL. For example, pocket or pockets may be arranged around the bottom surface of base section 20 and/or at least partially upward therefrom. Exemplary skirts S comprising pocket(s) P are shown in the Figures, with particular reference to FIGS. 12-15. As shown in FIGS. 13-15, subpockets P′ may be provided within the two-layered skirt to provide discrete locating of the hydrophilic material M at regions particularly susceptible or vulnerable to insufficient apposition and/or PVL. As shown in FIG. 14, at least some subpockets P′ may be formed and configured to swell radially outwardly to, inter alia, seal and in some cases partially fill the left atrial appendage or LAA.

In embodiments as in FIGS. 5-7 and 15-17, wherein the valve support 30, or boss 40 or other structure, extends at least partially outwardly from base section 20, the skirt S may cover at least part of the outer surface 34 of the valve support 30, with hydrophilic material M integrated or otherwise comprising the skirt S including but not limited to inclusion in pocket(s) P and/or subpockets P′ formed as described above.

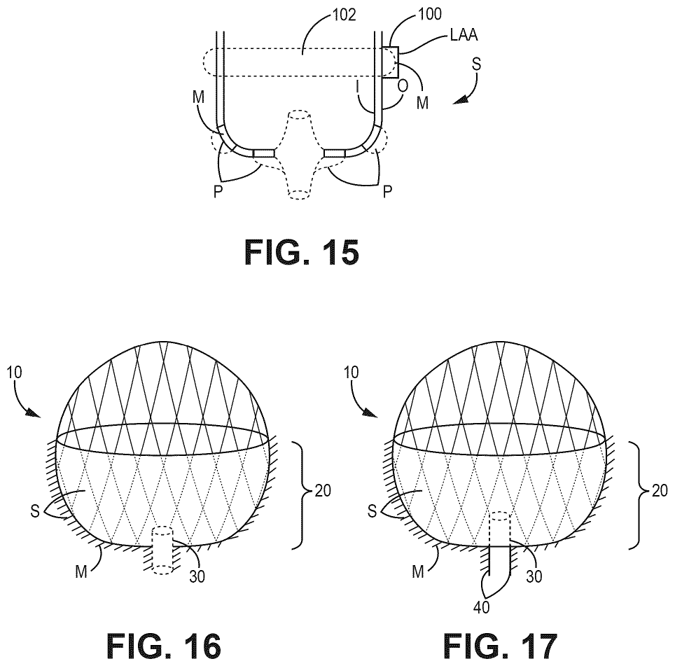

In the embodiment comprising a boss structure, or other extension into the inner throat of the annulus, pocket(s) P and/or subpockets P′ comprising material M may be formed between the boss structure and the base section 20 to swellingly close any gap between the device and the patient's anatomy. This is best shown in FIG. 15.

Alternatively, in the embodiments comprising encapsulated hydrophilic material M, the nanoparticles or capsules may be integrated, or incorporated into, or coated, attached or adhered to, the skirt in at least the PVL-vulnerable areas discussed above. Still more alternatively, the nanoparticles or capsules carrying hydrophilic material M may be affixed or adhered or coated onto or integrated into the skirt.

In certain embodiments, therefore, a pocket P formed in skirt S is not required and the skirt S may be formed of, or comprise, a single layer of material, with the hydrophilic material M affixed or adhered or coated thereon, or integrated therein, in either encapsulated or non-encapsulated forms. FIGS. 16 and 17 illustrate exemplary single layer skirts S comprising hydrophilic material M.

In alternative embodiments, portions of the anchoring structure 10 may be at least partially formed from hydrophilic material M and may be covered or overcoated by a thin film of biodegradable, dissolvable, biocrodable and/or bioabsorbable material to delay solute interaction, and resulting swelling, with hydrophilic material M. For example, and without limitation, boss structure 40, or other extension into the inner throat of the annulus, may comprise a hydrophilic polymer that swells when contacted with a solute, e.g., water within blood. In this embodiment, a biodegradable, bioerodable and/bioabsorbable thin coating layer may be applied over the boss structure 40 to appropriately delay swelling until after implantation is achieved. The outer portions of the boss structure 40, i.e., those portions that are juxtaposed by and/or within the annulus and/or inner throat of the annulus, may comprise the hydrophilic material, e.g., polymer, so that only the outer portion of boss structure 40 swells in response to solute contact, leaving the dimensions of the inner boss structure 40 unaltered. Other areas of the anchoring structure 10 may also be formed from hydrophilic material M, e.g., a polymer(s), e.g., key struts or cells of the anchor 10 may comprise hydrophilic polymer that swells on solute contact.

Further, portions of anchoring structure 10 may also comprise a skirt S comprising a thin film of hydrophilic material M that may also be covered, or overcoated, temporarily during delivery and implantation by a biodegradable, dissolvable, bioerodable and/or bioabsorbable thin film layer as described above.

Moreover, nanoparticles encapsulating hydrophilic material M as described above may be adhered or coated onto portions of anchoring structure 10 to comprise skirt S. These nanoparticles may be overcoated with a thin biodegradable, dissolvable, bioerodable and/or bioabsorbable thin film to ensure adherence to the anchoring structure during delivery and implantation.

Each of the possible embodiments described above for implementing hydrophilic skirt comprising or incorporating hydrophilic material M may be used to cover portions of various configurations of prosthetic heart valve devices. Exemplary embodiments wherein the valve support 30 is formed from, or otherwise integrated or attached with, base section 20 are shown in FIGS. 4A, 8-10, 11A and 16-17. In this case, hydrophilic skirt S comprising hydrophilic material M as discussed above may cover the outer surface 22 of base section 20, extending to cover the bottom of base section 20 and extending further upward within base section 20 to cover the inner surface 32 of valve support 30.

In some embodiments, sealing of the left atrial appendage (LAA) may be an objective. In these cases, as shown in FIG. 15, a hydrophilic skirt S comprising hydrophilic material M may comprise a reserve pocket 100 of hydrophilic material M in the region of the LAA, wherein upon implantation, the hydrophilic material M swells to enlarge pocket 100 to cover and/or fill the LAA. The hydrophilic material M reserve may comprise a ring or gasket 102 of material M around the circumference of the skirt S so that locating the LAA is achieved no matter the rotational position of the implanted anchoring structure 10. Alternatively, the specific pocket 102 may be provided as described above that is located to the LAA for swelling sealing and/or filling of the LAA. The hydrophilic material M reserve may be formed according to the various embodiments discussed herein, including pocket(s) and/or nanoparticles and/or coating.

Generally, the improved sealing and/or apposition may therefore be improved by including hydrophilic material M at one or more locations on an anchoring structure 10. The hydrophilic material M may be associated or integrated with or incorporated with a skirt S, but this is just one embodiment. Nanoparticles, when employed, may comprise an easily breakable material and/or biodegradable, bioerodable or dissolving material to provide the desired delay in exposing the hydrophilic material M to blood.

With reference now to FIGS. 4A-8, generally, it is to be noted that the embodiments illustrated are substantially symmetrical about a central axis A (as shown in FIG. 4A, for example).

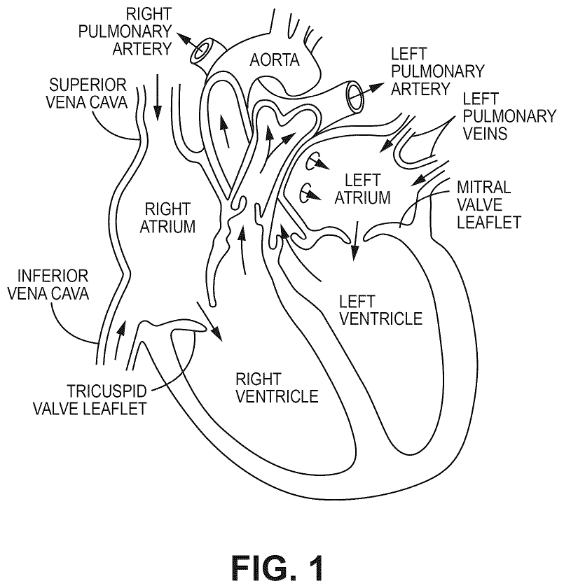

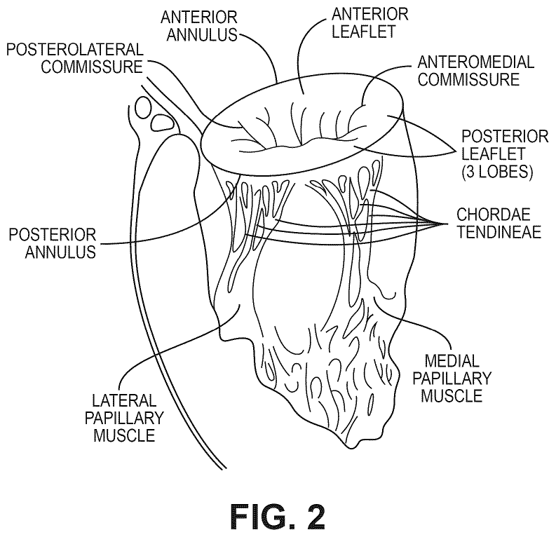

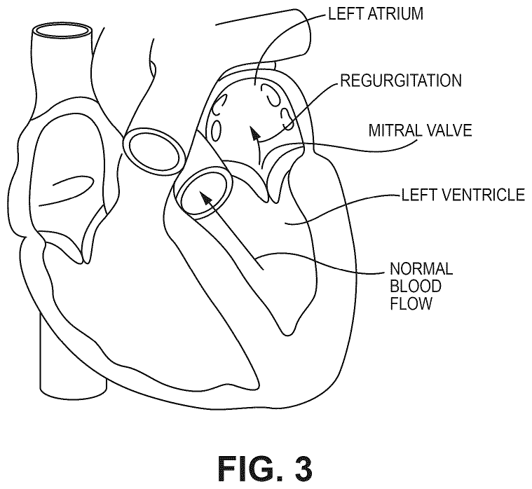

As noted above, the left atrium is not perfectly symmetrical. More specifically, the left atrium comprises an antior medial tilt. This anterior medial tilt, may lead in some cases to movement of an implanted device attempting to accommodate and/or adapt to the shape of the left atrial chamber. This asymmetry is best shown in FIGS. 1 and 18-20.

The devices shown in the FIGS. 4A-11, and 16-17 comprise a stent frame of a certain shape which is obtained, as the skilled artisan appreciates, by using a mandrel that has the desired shape. The stent frame material is stretched over the mandrel and processed by known means to achieve the desired stent frame shaping.

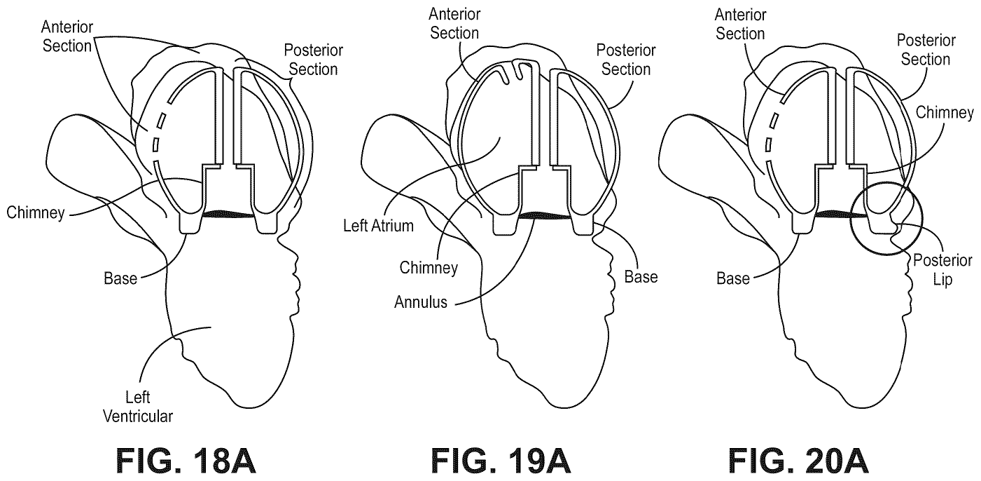

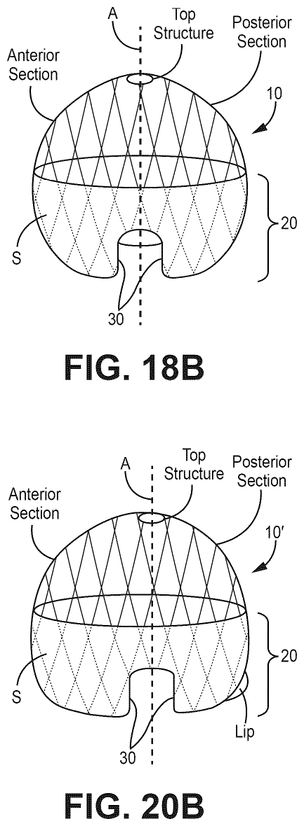

The stent mandrel of FIG. 18 is essentially a mandrel that results in the shape of, e.g., the symmetrical stent frame of FIG. 4A. The mandrel has substantially equally sized anterior and posterior sections, each of the anterior and posterior sections having substantially the same shape, wherein the “chimney” that forms the valve support of the prosthetic heart valve device is centrally located between the anterior and posterior sections, wherein the resulting valve support is arranged to be over the annulus when implanted. A top structure that may comprise a ring to aid in distributing forces around a greater area may be provided as shown. The mandrel is shown as oversized relative to the atrium which may be desired in order to provide a slightly oversized stent frame that, when expanded, generates strong frictional fit within the heart chamber.

The device of 18B thus comprises a central axis A with the posterior and anterior sections, valve support and top structure are symmetrically disposed in relation to axis A, and wherein the top structure and valve support are effectively aligned along axis A. Top structure may, or may not be present in the various embodiments.

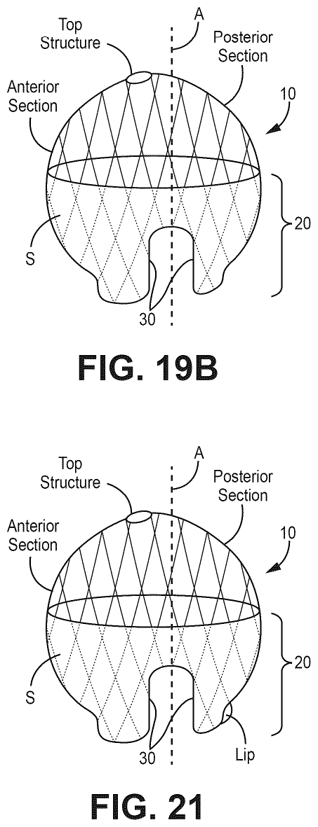

FIG. 19A provides one alternative solution wherein the mandrel comprises an asymmetric “bean” shape and wherein the “chimney” giving rise to the valve support in the resulting stent frame is, as in FIG. 18A, arranged to be over the annulus when the device is implanted. As illustrated in 19A, a posterior portion or section of the mandrel is smaller than the anterior portion or section. Additionally, the anterior portion or section is larger than the anterior portion of FIG. 18A, and the posterior portion or section is smaller than the posterior portion of FIG. 18A. FIG. 19B provides an exemplary device resulting from processing with mandrel of FIG. 19A.

As shown, the arrangement of FIG. 19A allows the anterior section to accommodate or engage more of the left atrium than the arrangement of FIG. 18A while still maintaining the fluid flow path through the valve support, down into the annulus and through the native valve leaflets.

The top structure of FIG. 19B, which may be optional, is now shown as shifted away from axis A and into the anterior section to aid in force transmission and/or distribution.

FIG. 20A comprises a mandrel that is the same as in FIG. 18A, with one difference: the posterior base section of the mandrel defines a radially extending lip. The resulting device, shown in FIG. 20B, will thus comprise a radially extending lip which, when expanded, will engage the left atrium on the posterior side of the chamber with more frictional gripping force than the device of FIG. 18B, thus aiding in shifting due to the asymmetry of the atrium after implantation. Top structure is shown as aligned with axis A, as in FIG. 18B.

A combination of the device of FIGS. 19B and 20B may be provided as shown in FIG. 21, wherein the frame portion is asymmetrical and the posterior lip structure is provided, and top structure is offset from axis A, with the valve support aligned with axis A and arranged to be positioned over the annulus when implanted. An alternative arrangement may not comprise the top structure, or may comprise the top structure also aligned with axis A.

In all cases, the “chimney” and resulting valve support, are arranged to be perpendicular to the base of the stent. As discussed further above, the length of the “chimney” and resulting valve support may vary and may, or may not, be entirely disposed within the interior of the stent frame.

In any of the described embodiments, the anterior section of the stent frame may comprise a hydrophilic material, e.g., a hydrophilic metal, that expands when exposed to liquid, e.g., blood. The hydrophilic metal may be adapted to expand only radially outwardly to enhance conformance of the anterior section to the chamber, e.g., left atrial, wall on the anterior side. Alternatively, the outer portion of at least a portion of the anterior section may be coated with a hydrophilic material that expands radially outwardly to achieve better conformance with the anterior atrial wall. Still more alternatively, some, or all, of the outer stent frame may comprise a hydrophilic material to enhance expanded conformance (and engagement) with the chamber wall. As described above, the hydrophilic material may be coated with a thin film, and/or encased in nanoparticles, to delay the hydrophilic expansion until the device is released from the delivery catheter into the heart chamber and at least provisionally positioned.

Similarly, the posterior lip of exemplary stent frame in FIG. 20B may be formed and/or defined in the device as a result of the stent frame taking on the mandrel's shape, which includes the lip structure. In this case, the lip is provided on the stent during delivery and implantation. Alternatively, the stent frame in the location of the desired lip may comprise a hydrophilic material, e.g., a hydrophilic metal, or may be coated with a hydrophilic material. The hydrophilic material, whether metal or a coating, may be covered with a thin film. The coating alternative may comprise the hydrophilic material encased in nanoparticles to allow the lip to form only after the device is positioned and implanted. Still more alternatively, the lip may be formed by a hydrophilic pocket as described above.

As shown, the lip is not symmetric around the device and is effectively a raised ridge or lip that extends radially outwardly away from a portion of the base of the prosthetic heart valve device. Alternative lip structures may surround the entire base structure, such that the lip is effectively symmetric.

Moreover, the raised lip may be defined on a portion of the base section that extends downwardly slightly into the annulus when implanted.

Still more alternatively, the raised lip may be defined on, or proximate, a portion of the base section that engages the upper surface of the annulus, a structure that may be referred to as an annular ring.

The raised lip therefore, may increase the oversizing of the base section to improve apposition, fit and prevent shifting movement on implantation.

The asymmetric structures discussed herein require a directional delivery technique to achieve the proper positioning of the device prior to implant. For example, the asymmetric stent frame embodiment of FIG. 19B requires that the posterior section be positioned against the posterior wall of the left atrium. Similarly, the posterior lip section of FIG. 20B will need to be delivered and/or positioned so that the lip is adjacent the posterior wall of the left atrium.

Accordingly, the lip structure may be provided as a consequence of treatment and processing with the mandrel as shown in the Figures. To help in positioning and implanting, the lip may, in some cases, be made smaller than desired, but also adapted to enlarge through hydrophilic expansion as discussed above, in order to tighten the expansion of the device against the posterior wall. In other cases, the lip structure may be entirely formed of a hydrophilic structure as discussed above. In this case, the lip structure only begins to form after the delivery of the prosthetic valve device into the wet chamber which may assist in keeping delivery profiles as low as possible as well as in recapturing and repositioning.

The hydrophilic expansion techniques described herein may be used, either alone or in combination with a mandrel, to create and/or modify the shape of the stent frame and/or lip structure and may be further used to create the lip structure.

The improved prosthetic heart valve devices described herein provide better apposition and fit against the chamber, e.g., left atrium, walls and annulus, thus (1) mitigating shifting of the implanted device as a result of the asymmetry of the heart chamber; (2) improving sealing against relevant tissue which, in turn, assists in preventing PVL

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1-8. (canceled)

9. A device for expanded implantation into a left atrium of a heart, the device comprising:
an expandable stent frame comprising:
a posterior section;
an anterior section;
a base section comprising a posterior section and an anterior section; and
a valve support adapted for supporting at least one prosthetic valve; wherein;
the posterior section and the anterior section of the expandable stent frame are asymmetrically shaped relative to each other;
a lip extends at least partially around a portion of the base section, the lip extending outwardly away from the base section; and
when the device is implanted and expanded:
the stent frame is configured to engage the left atrium;
the base section is configured to engage annular tissue of the left atrium; and
the lip is configured to engage the annular tissue of the left atrium.

10. The device of claim 9 wherein the lip is disposed on the posterior section of the base section.

11. The device of claim 9 wherein the lip is formed using a mandrel.

12. The device of claim 9 wherein;
the lip is formed at least in part from a hydrophilic material; and
when wetted, the lip is configured to extend radially outwardly away from the base section due to an expansion of the hydrophilic material.

13. (canceled)

14. The device of claim 9, further comprising a skirt of single layered material operatively attached to a portion of an outer surface of the base section;
wherein:
the skirt comprises hydrophilic material that is integrated into, or coated onto, the single layered material of the skirt to form the lip when wetted.

15-22. (canceled)

23. The device of claim 9 wherein the expandable stent frame further comprises a top structure.

24. The device of claim 23 wherein the top structure is disposed in the anterior section.

25. The device of claim 23 wherein:
the device defines a central axis; and
the top structure is spaced apart from the central axis.

26. The device of claim 25 wherein a central axis of the valve support extends along the central axis of the device.

27. The device of claim 23 wherein:
the device defines a central axis; and
the top structure is aligned with the central axis.

28. The device of claim 27 wherein a central axis of the valve support extends along the central axis of the device.

29. The device of claim 9 wherein a central axis of the valve support extends along a central axis of the device.

30. The device of claim 9 wherein the posterior section is smaller than the anterior section.

31. The device of claim 9 wherein, when the device is implanted in the left atrium:
the posterior section is positioned adjacent a posterior wall of the left atrium; and
the anterior section is positioned adjacent an anterior wall of the left atrium.

32. The device of claim 31 wherein, when the device is implanted in the left atrium, the anterior section engages more of the left atrium than the posterior section.

33. The device of claim 10 wherein, when the device is implanted in the left atrium, the lip engages a posterior side of the left atrium.

34. The device of claim 9 wherein the expandable stent frame defines the lip.

35. The device of claim 9 wherein the lip is asymmetric about the device.

36. The device of claim 9 wherein the lip extends radially outwardly away from the base section.

37. The device of claim 9 wherein the lip surrounds the entire base section.

38. The device of claim 9 wherein, when the device is implanted in the left atrium, the base section is positioned an annulus of the left atrium.

39. The device of claim 38 wherein, when the device is implanted in the left atrium, the lip is positioned in the annulus.
